(19) United States
(12) Patent Application Publication
(10) Pub. No.: US 2003/0054058 A1
(43) Pub. Date: Mar. 20, 2003

(54) Vitex agnus castus extract

(76) Inventors: David Gregory Corley, Prangins (CH); Qing Lu, Livingston, NJ (US); Ding Ming, Morris Plains, NJ (US); John P. Troup, Commugny (CH)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/140,582

(22) Filed: May 8, 2002

Related U.S. Application Data
(60) Provisional application No. 60/289,840, filed on May 9, 2001.

(51) Int. Cl. A61K 35/78; A61K 31/202

(52) U.S. Cl. 424/777; 514/560; 514/168; 514/350; 514/356

(57) ABSTRACT

The present invention provides a Vitex agnus castus extract wherein the extract is obtained by extracting dried and pulverized fruits of the plant Vitex agnus castus with a 90-100% ethanol solvent, separating the extraction solution from the rest of the plant material, removing the solvent from the extraction solution and recovering the extract. The present invention also provides for a dietary supplement comprising a Vitex agnus castus extract having a linoleic acid content of at least ten weight percent by the composition and a calcium source and the use of the extract and dietary supplement to treat conditions particularly affecting women.

### RELATED APPLICATION

This application claims the benefit of U.S. Provisional Application No. 60/289,840 filed on May 9, 2001.

### FIELD OF THE INVENTION

The present invention relates to extracts of Vitex agnus castus, and the use of such extracts for the management of premenstrual syndrome and various menopausal and post-menopausal disorders. The present invention also relates to dietary supplements comprising extracts of Vitex agnus castus.

### BACKGROUND OF THE INVENTION

The nutritional and health needs of women differ in many respects from those of men. In general, women pass through three principal adult developmental or life stages—the childbearing or pre-perimenopausal stage; the perimenopausal and menopausal stage; and the post-menopausal stage. Numerous health conditions and risks may develop during each of these life stages. They include pre-menstrual syndrome (PMS) and perimenopausal, menopausal and post-menopausal disorders including coronary heart disease (CHD) and osteoporosis.

PMS is a common recurring multi-symptom condition experienced by many menstruating women. Symptoms include water-retention, breast tenderness, headaches, lower back-aches, mood swings, etc.

Menopause can result in various unpleasant symptoms, including hot flashes, night sweats, mood swings, insomnia and fatigue.

CHD is a major cause of death in women. Although CHD generally does not manifest until the post-menopausal stage, CHD develops over decades.

Osteoporosis is associated with the aging process and predominantly affects women. It is characterized by diminished bone density, which results in increased bone fractures and vertebral column collapse. Bone loss begins around age 35. This loss accelerates during the menopause, which generally occurs around age 45 to 55. Osteoporosis develops over decades and is related to peak bone mass, as well as to the degree of bone loss. Adequate calcium intake can decrease the severity of osteoporosis.

Hormone replacement therapy (HRT) has been shown to be useful in the treatment and amelioration of several estrogen-related conditions including, but not limited, to bone loss, e.g. osteoporosis, hot flashes, sleep disorders, mood, and the collective symptoms of PMS. Current therapies involve the oral intake of estrogen, selective estrogen receptor modulators (SERMS) and phytoestrogens, e.g. soy isoflavones. Each of the current therapies involve the direct interaction with the estrogen receptor (ER).

Currently there are several products available and described in the literature that use the dried ground fruits or a 60% ethanolic extract of the fruits of Vitex agnus-castus to alleviate the symptoms of PMS.

### SUMMARY OF THE INVENTION

The present inventors have now found that an extract of the fruits of the Vitex agnus castus plant interacts directly with the ER and consequently provides symptomatic relief of PMS, peri- and postmenopausal symptoms. The extract of the current invention shows a marked improvement over existing methods. The extraction method used by the present inventors leads to a Vitex agnus castus extract which is enriched in linoleic acid and which is significantly more active against the estrogen receptor, both subtypes ER-α and ER-β, when compared to currently available Vitex agnus castus products. ER-α is found predominantly in bone, cardiovascular, breast and reproductive tissues, while ER-β is the predominantly expressed estrogen receptor in the prostate and brain.

In one aspect of the present invention there is provided an improved Vitex agnus castus extract wherein the extract is obtainable by extracting dried and pulverized fruits of the plant Vitex agnus castus with a 90-100% ethanol solvent, separating the extraction solution from the rest of the plant material, removing the solvent from the extraction solution and recovering the extract. The extract so recovered may be further purified, e.g. by way of suitable extraction procedures. However, preferably the extract is used as a crude extract.

In a further aspect of the present invention there is provided an improved Vitex agnus castus extract wherein the extract is obtained by extracting dried and pulverized fruits of the plant Vitex agnus castus with a 90-100% ethanol solvent to make an extraction solution, and removing the solvent from the extraction solution to form the extract.

Extracts prepared by this method preferably have a linoleic acid content of at least 10 weight % by weight of the extract, more preferably at least 15 weight %, even more preferred at least 20 weight %. Conventionally prepared extracts, e.g. 60:40 ethanol/water extracts, have a linoleic acid content of less than 10 wt %.

In another embodiment of this aspect of the invention there is provided the method of controlling, e.g. treating and/or preventing, symptoms of pre-menstrual syndrome (PMS) and/or peri- and/or postmenopausal disorders including coronary heart disease (CHD) and osteoporosis by administering to a subject in need of such treatment an effective amount of the improved Vitex agnus castus extract defined above.

In another aspect of the present invention there is provided a dietary supplement for women comprising 10 to 100 parts by weight, preferably 20 to 80 parts by weight, more preferably 15-50 parts by weight, of a Vitex agnus castus extract having a linoleic acid content of at least 10 wt %; 50 to 225 parts by weight, preferably 100 to 200 parts by weight, more preferably 120-180 parts by weight, of a calcium source; and a physiologically acceptable carrier.

Preferably, the supplement further comprises one or more of the following ingredients:

50 to 250 parts by weight, preferably 100 to 200 parts by weight, more preferably 120-180 parts by weight, of a source of DHA or a DHA/EPA mixture; 50 to 500 parts by weight, preferably 20 to 150 parts by weight, more preferably 50-100 parts by weight, of a magnesium source;

0.1 to 200 parts by weight of vitamin B₆, preferably 0.5 to 100 parts by weight, more preferably 1-20 parts by weight; and/or 0.00025 to 0.001 parts by weight, preferably 0.0025 to 0.0075 parts by weight, of a source of vitamin D, wherein DHA indicates docosahexaenoic acid and EPA indicates eicosapentaenoic acid.

Hereinafter the Vitex agnus castus extract; calcium; DHA and/or the DHA/EPA mixture; vitamin D; vitamin B₆; and/or magnesium are referred to as active ingredients.

For calcium and magnesium the parts by weight are referred to on a elemental basis. For DHA and EPA the parts by weight are referred to on a pure substance basis.

In one embodiment of this aspect of the invention there is provided a method of controlling, e.g. treating and/or preventing, symptoms of pre-menstrual syndrome (PMS) and/or peri- and/or postmenopausal disorders including coronary heart disease (CHD) and osteoporosis in an adult women by administering to a woman in need of such treatment an effective amount of the dietary supplement defined above.

### DETAILED DESCRIPTION OF THE INVENTION

For the extraction process the ratio solvent/plant material is preferably 10:1, but can range from 2:1 to 100:1. For obtaining a Vitex agnus castus extract having a linoleic acid content of at least 10 wt % solvents can range from any solvent that is more “lipophilic” than 60% ethanol including 90-100% ethanol, acetone, ethyl acetate, butanol, isopropanol, methanol, methyl isobutyl ketone, chloroform, dichloromethane, carbon tetrachloride. The extraction conditions are typically stirring at room temperature for 4 hours, but can range from 1 second to several days and at a temperature between the freezing and boiling points of the solvent and under normal pressure, super-critical pressure or near super-critical pressure. Methods for separating the extraction solution from the plant material include standard filtration by gravity or reduced pressure and methods of removing solvent include reduced pressure evaporation or normal temperature-driven evaporation.

Daily dosage of the extract typically range between 10 mg to 100 mg of Vitex agnus-castus extract, preferably between 20 to 80 mg of Vitex agnus-castus extract and more preferably between 15 to 50 mg of Vitex agnus-castus extract.

Suitable calcium sources may comprise any physiological acceptable inorganic or organic compound containing calcium. Examples include, but are not limited to, inorganic calcium salts, for example calcium chloride, calcium phosphate, calcium sulfate, calcium oxide, calcium hydroxide or calcium carbonate, or organic calcium components like whole or skim milk powder, calcium caseinate or calcium salts of organic acids such as calcium citrate, calcium maleate, or mixtures thereof. The use of organic calcium compounds, particularly skim milk powder, calcium caseinate or mixtures thereof, as calcium source is preferred. The amount of calcium to be supplied may vary within wide ranges. In general, the dietary supplement comprises in one unit dosage from about 50 mg to 225 mg, preferably 100 mg to 200 mg and more preferred 120 to 180 mg of calcium (on an elemental basis).

Suitable EPA and DHA sources include fish oils such as menhaden oil, salmon oil, mackerel oil, tuna oil cod liver oil and anchovy oil, highly refined egg yolk oil, macroalgae oil, e.g. from seaweed types, and microbial oils, i.e. those oils naturally produced by microorganisms during their lifespan. The amount of DHA or DHA/EPA mixture to be supplied may vary within wide ranges. In general, the dietary supplement may comprise in one unit dosage from about 50 mg to 250 mg, preferably 100 mg to 200 mg and more preferred 120 to 180 mg of DHA or a DHA/EPA mixture (on a pure substance basis).

Suitable magnesium sources may comprise any physiological acceptable inorganic or organic compound containing magnesium such as magnesium gluconate, magnesium oxide, magnesium citrate or magnesium lactate. The amount of magnesium to be supplied may vary within wide ranges. In general, the dietary supplement may comprise in one unit dosage from about 50 mg to 500 mg, preferably 150 mg to 300 mg and more preferred 175 to 250 mg of magnesium (on an elemental basis).

The amount of vitamin B₆ (pyridoxine) to be supplied may vary within wide ranges. In general, the dietary supplement may comprise in one unit dosage from about 0.1 mg to 200 mg, preferably 0.5 mg to 100 mg and more preferred 1.0 to 20 mg of vitamin B₆.

Suitable vitamin D sources include vitamin D₃ (cholecalciferol). The amount of vitamin D to be supplied may vary within wide ranges. In general, the inventive compositions may comprise in one unit dosage from about 10 IU to 400 IU, preferably about 100-300 IU.

Preferably the unit doses are taken once daily without restriction to time of day.

The dietary supplements are intended for enteral administration, such as oral or nasal administration. Suitable pharmaceutical compositions may be in liquid form or in solid form, preferably in solid form, and comprise (in % by weight) for example, from approximately 0.001% to 100%, preferably from approximately 0.1 to approximately 50%, active ingredients.

Dietary supplements for enteral administration are, for example, those in single dose unit forms, such as dragées, tablets, capsules or sachets. They are prepared in a manner known per se, for example by means of conventional mixing, granulating, confectioning, dissolving or lyophilising processes.

For example, dietary supplements for oral administration may be obtained by combining the active ingredients with solid carriers, optionally granulating a resulting mixture and processing the mixture or granules, if desired or necessary after the addition of suitable excipients, to form tablets or dragée cores.

Suitable physiologically acceptable carriers may be especially fillers, such as sugars, for example lactose, saccharose, mannitol or sorbitol, cellulose preparations and/or calcium phosphates, for example tricalcium phosphate or calcium hydrogen phosphate, and also binders, such as starch pastes using, for example, corn, wheat, rice or potato starch, gelatin, tragacanth, methylcellulose and/or polyvinylpyrrolidone, and, if desired, disintegrators, such as the above-mentioned starches, and also carboxymethyl starch, cross-linked polyvinylpyrrolidone, agar, or alginic acid or a salt thereof, such as sodium alginate. Further excipients may be especially flow-conditioners and lubricants, for example silicic acid, talc, stearic acid or salts thereof, such as magnesium or calcium stearate, and/or polyethylene glycol. Dragée cores are provided with suitable, optionally enteric, coatings, there being used inter alia concentrated sugar solutions which may contain gum arabic, talc, polyvinylpyrrolidone, polyethylene glycol and/or titanium dioxide, or coating solutions in suitable organic solvents or solvent mixtures or, for the preparation of enteric coatings, solutions of suitable cellulose preparations, such as acetylcellulose phthalate or hydroxypropylmethylcellulose phthalate. Dyes or pigments may be added to the tablets or dragée coatings, for example for identification purposes or to indicate different doses of active ingredient.

Other orally administrable dietary supplements may be in the form of hard gelatin capsules or soft, sealed capsules consisting of gelatin and a plasticizer, such as glycerol or sorbitol. The hard gelatin capsules may comprise the active ingredient in the form of granules, for example in admixture with fillers, such as lactose, binders, such as starches, and/or glidants, such as talc or magnesium stearate, and, if desired, stabilizers. In soft capsules the active ingredient is preferably dissolved or suspended in suitable liquids, such as fatty oils, paraffin oil or liquid polyethylene glycols, it is likewise being possible to add stabilizers.

The invention will now be further illustrated by the following examples.

### EXAMPLES

### Example 1

### 95% EtOH Extract of Vitex Agnus Castus

1. Place 10 g of the Vitex agnus-castus fruit powder into a 125-ml Erlenmeyer flask.

2. Add 100 ml of 95% ethanol (Fisher Scientific, Cat#: A405-20) into the flask.

3. Cover the flask with a watch glass to eliminate solvent vapor loss.

4. Stir the mixture in the flask at room temperature (˜25° C.) for 15 hours (overnight).

5. Separate the extract solution from the material by filtration.

6. Remove the solvent of the extract by rotary evaporation and then high vacuum pumping.

7. Transfer the EtOH extract to a vial for bioassays.

The biomarker percentages in the extracts of Vitex agnus-castus:

| | Linoleic acid % | Casticin % | Agnuside % |
|---|---|---|---|
| 95% EtOH extract of | 20.98 | 1.13 | 0.092 |
| Vitex agnus-castus | | | |
| 60% EtOH extract of | 8.4 | 0.74 | 0.24 |
| Vitex agnus-castus | | | |

### Example 2

### Estrogen Receptor Binding Assay

The 95% EtOH extract of Vitex agnus-castus prepared in Example 1 and a standard 60% EtOH extract of Vitex agnus-castus are tested for their ER binding activity. For this assay Greiner Medium Binding black 96 well plates are used. Total volume of assay is 100 μl.

The two extracts are diluted in 25% DMSO/ES2 buffer in an intermediate plate. The dilution found to be most appropriate for testing ER binding activity was 500×. A 50×dilution is performed in the intermediate plate by adding 5 μl of the 20 mM stock compound in 100% DMSO to 245 μl DMSO/ES2 buffer. This creates a 250 μl total volume in the well. Then 10 μl of this dilution is added to the black test plate. This further dilutes the sample 10×to a final 500×dilution.

As controls 10 μl of the non-specific inhibitor diethylstilbestrol (DES) in a 100 μm of DES in 5% DMSO and 5% DMSO are used.

ER Alpha: a dilution is prepared to achieve a 15 nM final concentration in the test well. 45 μl of diluted ER Alpha are added to each well. A total of 90 μl will be needed for each well when mixed 1:1 with the tracer ES2 Fluormone.

ER Beta: a dilution is prepared to achieve a 10 nM final concentration in the test well. 45 μl of diluted ER Beta is added to each well. A total of 90 μl will be needed for each well when mixed 1:1 with the tracer ES2 Fluormone.

The ER, either alpha or beta is added without the presence of ES2 Fluormone to the four ER control wells already containing 10 μl of 5% DMSO.

The tracer ES2 Fluormone, (Fluorescein 400 nM stock), is prepared in a 1:360 dilution to achieve a 1 nM final concentration in each well.

Total volume in the wells should be 100 μl.

The plate should be mixed by shaking on a plate shaker and incubated in the dark at room temperature for 2 hours. The plate is read on a LJL analyst using Excitation/Emission filters of 485/530 nM at a z height of 5.35 mm with an integration time of 100 msec.

TABLE 1
Result of Estrogen Receptor Binding Assay:

| IC50's | ER alpha | ER beta |
|---|---|---|
| 95% EtOH | 1.98 ug/ml | 2.14 ug/ml |
| 60% EtOH | 5.98 ug/ml | 6.02 ug/ml |

Obviously, the overall ER binding activity in 95% EtOH is roughly about 3X as in 60% EtOH extract, either in ER alpha or ER beta.

What is claimed is:

1. A Vitex agnus castus extract comprising an extract obtained by a process which comprises the steps of:
extracting fruits of the plant Vitex agnus castus with a 90-100% ethanol solvent to make an extraction solution, and
removing the solvent from the extraction solution to form said extract.

2. The extract of claim 1 wherein said fruits are dried and pulverized.

3. The extract of claim 1 wherein said extract has a linoleic acid content of at least 10% by weight of the extract.

4. The extract of claim 1 wherein said extract has a linoleic acid content of at least 15% by weight of the extract.

5. A dietary supplement for women comprising
10 to 100 parts by weight of a Vitex agnus castus extract having a linoleic acid content of at least 10 wt %,
50 to 225 parts by weight of a calcium source,
and a physiologically acceptable carrier.

6. The dietary supplement of claim 5 further comprising a fatty acid selected from the group consisting of docosahexanoic acid, eicosapentaenoic acid and mixtures thereof.

7. The dietary supplement of claim 5 further comprising a magnesium source.

8. The dietary supplement of claim 5 further comprising vitamin B₆.

9. The dietary supplement of claim 5 further comprising vitamin D or derivative thereof.

10. A method of controlling symptoms of pre-menstrual syndrome and/or peri- and/or postmenopausal disorders including coronary heart disease and osteoporosis in an adult women by administering to a woman in need of such treatment an effective amount of the extract as claimed in claim 1.

11. A method of controlling symptoms of pre-menstrual syndrome and/or peri- and/or postmenopausal disorders including coronary heart disease and osteoporosis in an adult women by administering to a woman in need of such treatment an effective amount of the dietary supplement as claimed in claim 5.

12. A process for producing an Vitex agnus castus extract comprising the steps of
extracting dried and pulverized fruits of the plant Vitex agnus castus with a 90-100% ethanol solvent to form an extraction solution,
separating the extraction solution from the rest of the plant material to form an extract solution, and
removing the solvent from the extraction solution and recovering the extract.

13. A Vitex agnus castus extract wherein the extract is obtained by extracting dried and pulverized fruits of the plant Vitex agnus castus with a 90-100% ethanol solvent to make an extraction solution, separating the extraction solution from the rest of the plant material, removing the solvent from the separated extraction solution to form the extract, and recovering the extract.
